(19) United States
(12) Patent Application Publication
(10) Pub. No.: US 2004/0147043 A1
(43) Pub. Date: Jul. 29, 2004

(54) Microfluidic system for the manipulation and concentration of particles suspended in liquid

(76) Inventors: Gerben Boer, St-Blaise (CH); Arash Dodge, Neuchatel (CH); Gianluca Lettieri, Neuchatel (CH); Elisabeth Verpoorte, Neuchatel (CH)

Correspondence Address: FISH & RICHARDSON, PC, 12390 EL CAMINO REAL, SAN DIEGO, CA, 92130-2081, US

(21) Appl. No.: 10/475,553

(22) PCT Filed: May 13, 2002
(86) PCT No.: PCT/EP02/05324

(30) Foreign Application Priority Data
May 11, 2001 (GB) 011503.9

(51) Int. Cl. G01N 33/558

(52) U.S. Cl. 436/514

(57) ABSTRACT

A microfluidic system for the concentration of particulate matter comprises a first reservoir (1) containing a buffer solution, a second reservoir (2) containing a analyte, and a third reservoir (3) containing beads suspended in a liquid. Microchannels link the reservoirs and an expanded portion (4). Hydrostatic pressure is applied to the reservoir (3) containing the beads while an electro-osmotic force (EOF) is applied between the reservoir (1) and the reservoir (3) to establish counter flows of liquids. A vortex forms in the flared portion (5) due to the counter flow of the liquids and the beads are concentrated in the vortex. By switching the EOF between reservoirs (1) and (2) the buffer solution can be replaced by the analyte to enable an analysis to be performed.

### SUMMARY OF THE INVENTION

The present invention relates to a microfluidic system for the concentration of particulate matter and may be applied to a microfluidic system capable of accumulating and retaining large molecules or small beads in specific locations on a chip and perfusing them with liquids containing analytes, analyte/marker combinations, washing buffers etc. To achieve this, the invention provides a method and apparatus for locating particles in a vortex formed under pressure/electro-osmotic counterflow conditions.

The invention may be used, for example, for immunoassay or nucleic acid hybridisation based bioanalysis. In a preferred aspect of the invention opposing electro-osmotic and pressure driven flows are established in one or more capillaries such that liquid is flowing in one direction close to the walls and in the other direction at the centre of the capillaries. The capillaries have expanded sections at locations where the cross-sections increase. As a result, vortices are established which define the locations for particle accumulation and retention. In a preferred embodiment, a particle-loaded liquid is made to enter the system from the elevated pressure side and other liquids are made to enter from the electroosmotic driving side. Several inlets are provided on the electroosmotic driving side into channels that are joined at an intersection before reaching the particle accumulation locations, such that the flow can be switched between different locations by changing the applied voltages at the inlets. For the bioanalysis, the particles may advantageously be functionalised with sensing molecules and perfused with any succession of buffer and sample liquids containing the analyte, fluorescent sample containing the analytes, fluorescently marked molecules, other specifically binding molecules in any desired succession and combination. Arrays and networks of said structures are also straightforward generalizations of the invention. Reference should be made to the appended independent claims defining aspects of the invention. Preferred or advantageous features of the invention are set out in dependent subclaims.

In a preferred aspect, the invention provides a microfluidic system for the manipulation and concentration of beads suspended in liquid for bioanalysis with heterogeneous assay.

### DESCRIPTION OF EMBODIMENTS OF THE INVENTION BY WAY OF EXAMPLE

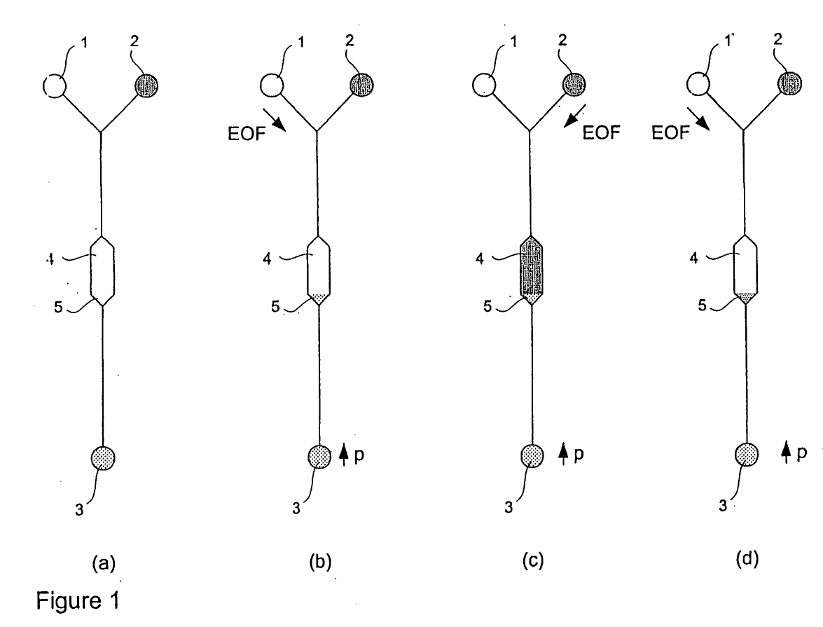

FIG. 1 illustrates an embodiment of apparatus used and steps performed in a preferred embodiment of the invention and particularly shows how an immunoreaction is performed. From this embodiment, it is easy for any one skilled in the art to devise more elaborate immunoassay formats well known in the state of the art, for example competitive assay formats or sandwich assay formats, and to devise more complex automated apparatus.

FIG. 1(a):

The system consists of a reservoir 1 containing a buffer solution, a reservoir 2 containing fluorescently marked molecules, and a reservoir 3 containing functionalised beads in a buffer solution. The reservoirs are connected via capillaries. An expanded section 4 is present in the capillaries with a flared area 5 where vortices are formed under pressure/EOF (electro-osmotic flow) counterflow conditions. In order to add or remove beads from the device, the reservoirs may be filled with suitable solutions or suspensions of beads. With simple apparatus the reservoirs may be filled or emptied by pipetting while more complex automated systems may include appropriate valves, sources of solutions, beads, etc, and drains.

FIG. 1(b):

A hydrostatic pressure is applied to reserovir 3, and an EOF force is applied to reservoir 1 by applying a high voltage between reservoir 1 and reservoir 3. This causes the beads to be transported with the flow from the reservoir 3 to the vortex region 5 where they are concentrated by the vortex in the flows from the reservoirs 1 and 3.

FIG. 1(c):

The EOF force is switched from reservoir 1 to reservoir 2. This causes analyte from the reservoir 2 to flow through the expanded section 4 and marked molecules bind (undergo an immunoreaction) to the beads that are clustering in the vortex region 5.

FIG. 1(d):

The EOF force is switched back from reservoir 2 to reservoir 1. This flushes the analyte solution from the expanded section 4 into the reservoir 3, leaving only the fluorescence due to the marked molecules on the beads. Once the analysis is complete the beads may be flushed into the reservoir 3 under EOF flow by removing the hydrostatic pressure flow.

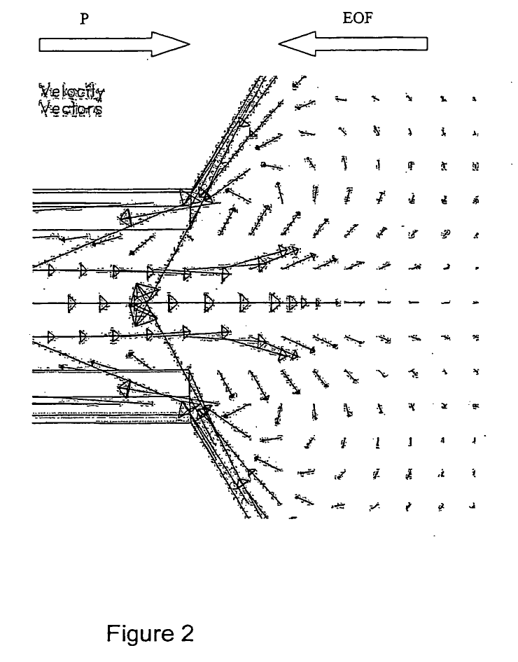

FIG. 2 illustrates the flow pattern at the vortex region 5 of FIG. 1 by showing the velocity vector field of the flow taken in a plane midway between the top and bottom of the expanded section. The direction of the arrows gives the direction of the streamlines while the sizes of the arrows indicate the relative magnitudes of the associated velocities at the points at which the arrows originate. In the centre of the cross section, the pressure driven flow is from left to right; near the walls, the EOF driven flow is from right to left. The combination of the two generates vortices.

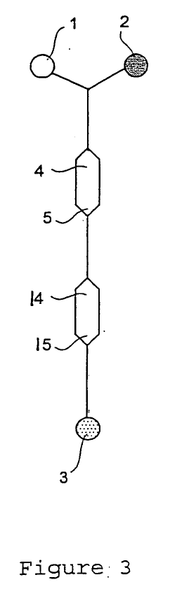

FIG. 3 illustrates a similar embodiment to that shown in FIG. 1 that is modified by providing two expanded portions 4 and 14 with flared areas 5 and 15 respectively. This allows the formation of two vortices for the concentration of beads and enables two analyses to be carried out simultaneously.

It is , of course, possible to provide further capillaries with further expanded portions to enable parallel analyses to be carried out, with or without the provision of valve means to enable a single reservoir to serve more than one capillary.

Further it is possible to reverse the flows due to pressure and electro-osmotic force, i.e. the buffer solution or analyte may be caused to flow by hydrostatic pressure while suspended beads are caused to flow by EOF. This applies both to the embodiment of FIG. 1 and that of FIG. 3.

### Prior Art

The following prior art is incorporated herein by reference.

The formation of vortices and the retention of beads by the use of electro-osmotic and pressure-generated forces acting in opposite directions has been described in G. Boer et al., in Micro Total Analysis Systems, ed. D. J. Harrison and A. van den Berg, Kluwer 1998, pp 53-56.

WO 00/70080, “Focusing of microparticles in microfluidic systems” teaches how to direct particles to a confined area but not how to increase their concentration nor to retain them in an area while maintaining an overall flow of the carrier liquid.

WO 00/50172 “Manipulation of microparticles in microfluidic systems” teaches how to perform analysis with particles perfused by liquids but with the particles retained by physical obstacles, leading to a number of disadvantages.

Methods to accumulate and retain particles in microsystems by physically blocking their path are well known, and for example described in B. Willumsen et al., Anal. Chem. 1997, 69, 3482-3489; R. Oleschuk et al., Anal. Chem. 2000, 72, 585-590; M. Mayer et al., Anal. Chem. 1996, 68, 3806-3814; K. Sato et al., Anal. Chem, 2000, 72, 1144-1147; H. Andersson et al., Micro Total Analysis Systems, 2000, 473-476. Such methods do not allow one to remove or otherwise manipulate the beads after they have been positioned. Furthermore, they are limited to beads above a certain size, typically several micrometers.

### Advantages

Bead-based materials have become omnipresent in applications like immunoassays, as they are ideal reagent delivery vehicles and provide high reactive surface areas. Specific advantages of various aspects of the present invention are mentioned hereinafter.

Preconcentration of beads in microchannels without micromachined barriers (formation of clusters)

Preconcentration of molecular species in microchannels

Bead handling (beads can be precisely moved from one point within a microfluidic system to another one)

Bead clusters may be held in place in a particular flow pattern while being sequentially perfused by different solutions. Conversely, beads may be transported into domains where molecular species have been concentrated.

Once an assay has been finished, the used beads may be easily removed from the device, and fresh beads brought in. This is achieved by flushing the beads to a drain reservoir and removing them. The drain reservoir can then become a source reservoir by loading it with new beads for a subsequent analysis. The reservoir alternates between a source and a drain reservoir by applying or removing hydrostatic pressure to or from it. Alternatively, separate drain and source reservoirs may be provided to enable fresh beads to be loaded and used beads to be extracted.

Clusters may be formed at multiple diffuser elements (that is expanded sections)simultaneously, opening a route to multistep analysis and multiple analyses on a single device.

The present invention may be used in applications ranging from diagnostics to DNA analysis, drug discovery.

1. A microfluidic system for the concentration of particulate matter wherein voltage and pressure differences are applied across liquid channels, where said voltage and pressure differences generate forces on the liquid in opposing direction, with the cross section increasing and decreasing over a short distance at some locations along the channels where vortices occur in the liquid flow within which particles are retained, and wherein said particles are retained in the vortex area or between vortex areas in increased concentration by an appropriate predetermined combination of said voltage and pressure differences.

2. A microfluidic system as in claim 1, wherein the particle size is between 1 nm and 10 μm.

3. A microfluidic system as in claim 1 or 2, wherein the particle size is between 100 nm and 10 μm.

4. A microfluidic system as in any of claims 1 to 3, characterized in that it comprises an array of channels having said vortex generating structures.

5. A microfluidic system as claimed in any of claims 1 to 4 in which a plurality of reservoirs are provided from which a buffer liquid and at least one analyte liquid may be selectively caused to flow through the channel by applying an electro-osmotic force to a selected reservoir.

6. A biochemical analysis method using a microfluidic system as in any of claims 1 to 5, wherein said particles are functionalised with a sensing molecule where a branching in the channel exists on the lower pressure side and said second liquid supply is switchable from a buffer liquid reservoir to a liquid reservoir containing an analyte.

7. A biochemical analysis method according to claim 6, wherein particles are first accumulated in the vortex region with one set of pressure/electrical parameters and then displaced as a cluster with a second set of pressure/electrical parameters to a different location in the channel system.

8. A biochemical analysis method according to claims 6 or claim 7 wherein when an analysis has been completed the particles are flushed into a reservoir by removing the hydrostatic pressure.

9. A biochemical analysis method according to claims 6 or claim 7 wherein when an analysis has been completed the particles are flushed into a reservoir by removing the EOF force.

10. A biochemical analysis method according to any of claims 6 to 9 wherein a plurality of vortices are produced in a plurality of channels to enable multiple analysis to be performed simultaneously.

11. A method of locating particles in a vortex comprising the steps of
causing a first solution to be passed along a first capillary to a section having a larger cross section joined to the capillary by a flared section by an electro-osmotic force
causing a second solution carrying particles to be passed along a second capillary to the section having a larger cross section by hydrostatic pressure, the flow of the first solution being in the opposite direction to that of the second solution, and
maintaining the particles in the flared section by selecting values of the electro-osmotic force and hydrostatic pressure to cause a vortex to be formed in the flared section to trap the particles within the vortex.
